(19) United States
(12) Patent Application Publication
(10) Pub. No.: US 2013/0158566 A1
(43) Pub. Date: Jun. 20, 2013

(54) ROTATIONAL MECHANISM FOR ENDOSCOPIC DEVICES

(71) Applicant: BOSTON SCIENTIFIC SCIMED, INC., Maple Grove, MN (US)

(72) Inventors: Colby HARRIS, Weston, MA (US); Gary JORDAN, Litchfield, NH (US)

(73) Assignee: BOSTON SCIENTIFIC SCIMED, INC., Maple Grove, MN (US)

(21) Appl. No.: 13/713,549

(22) Filed: Dec. 13, 2012

Related U.S. Application Data
(60) Provisional application No. 61/576,159, filed on Dec. 15, 2011.

(51) Int. Cl. A61B 17/08; A61B 17/00

(52) U.S. Cl. CPC A61B 17/08; A61B 17/00234
USPC 606/142

(57) ABSTRACT

A medical device includes a flexible member extending from a handle at a proximal end to a distal end sized and shaped for insertion to a target position in a living body and having a first channel extending longitudinally therethrough in combination with an elongated rotation tube positioned in the first channel and being rotatable relative to the flexible member and a rotation drive device at a distal end of the rotation tube, a proximal end of the rotation drive device being permanently attached to the rotation tube, a distal end of the rotation drive device having a first protrusion extending distally therefrom and a drive element having a substantially cylindrical shape with a groove on an inner wall thereof mating with the first protrusion of the rotation drive device, the drive element configured to transmit rotational movement of the rotation tube to an end effector connected to a distal end thereof to rotate the rotation tube relative to the flexible member.

### PRIORITY CLAIM

This present application claims the priority to the U.S. Provisional Application Ser. No. 61/576,159, entitled “Rotational Mechanism For Endoscopic Devices” filed on Dec. 15, 2011. The specification of the above-identified application is incorporated herewith by reference.

### BACKGROUND

Pathologies of the gastro-intestinal (“GI”) system, the biliary tree, the vascular system and other body lumens are commonly treated through endoscopic procedures, many of which require active and/or prophylactic hemostasis to control internal bleeding. Tools for deploying hemostatic clips via endoscopes are often used to control internal bleeding by clamping together the edges of wounds or incisions.

In the simplest form, these clips grasp tissue surrounding a wound bringing edges of the wound together to allow natural healing processes to close the wound. Specialized endoscopic clipping devices are used to deliver the clips to desired locations within the body and to position and deploy the clips at the desired locations after which the clip delivery device is withdrawn, leaving the clip within the body. Endoscopic hemostatic clipping devices are generally designed to reach tissues deep within the body (e.g., within the GI tract, the pulmonary system, the vascular system or other lumens and ducts) via a working lumen of an endoscope. Thus, the dimensions of such clipping devices are limited by the dimensions of endoscopic working lumens.

### SUMMARY OF THE INVENTION

The present invention relates to a medical device, comprising a flexible member extending from a handle at a proximal end to a distal end sized and shaped for insertion to a target position in a living body and having a first channel extending longitudinally therethrough in combination with an elongated rotation tube positioned in the first channel and being rotatable relative to the flexible member and a rotation drive device at a distal end of the rotation tube, a proximal end of the rotation drive device being permanently attached to the rotation tube, a distal end of the rotation drive device having a first protrusion extending distally therefrom and a drive element having a substantially cylindrical shape with a groove on an inner wall thereof mating with the first protrusion of the rotation drive device, the drive element configured to transmit rotational movement of the rotation tube to an end effector connected to a distal end thereof to rotate the rotation tube relative to the flexible member.

### BRIEF DESCRIPTION OF THE DRAWINGS

FIG. 1 shows a first perspective view of a device according to a first exemplary embodiment of the present invention in a first operative configuration;

FIG. 2 shows an exploded view of a distal rotation mechanism of the device of FIG. 1; and

FIG. 3 shows a perspective view of a proximal rotation mechanism of the device of FIG. 1.

### DETAILED DESCRIPTION

The present invention may be further understood with reference to the following description and the appended drawings, wherein like elements are referred to with the same reference numerals. The present invention relates to a device permitting rotation of an end effector of a flexible endoscopic device during insertion of the end effector to a target site within a living body. For example, one embodiment of the invention is directed to a hemostasis clipping device for applying one or more hemostatic clips, the device permitting rotation of the clip(s) during insertion of the clip to a target site in a living body. It is noted that although the exemplary device according to the invention has been described with respect to clipping devices, the device may also be designed to perform any of a variety of endoscopic procedures including, but not limited to, band ligation, injection therapy, thermal electrohemostasis, combination therapy needle, biopsies and fine-needle aspiration. The exemplary device according to the present invention is formed as an elongated member extending from a handle at a proximal end to an end effector (e.g., a clipping device) at a distal end. It should be noted that the terms “proximal” and “distal,” as used herein, are intended to refer to a direct toward (proximal) and away from (distal) a user of the device.

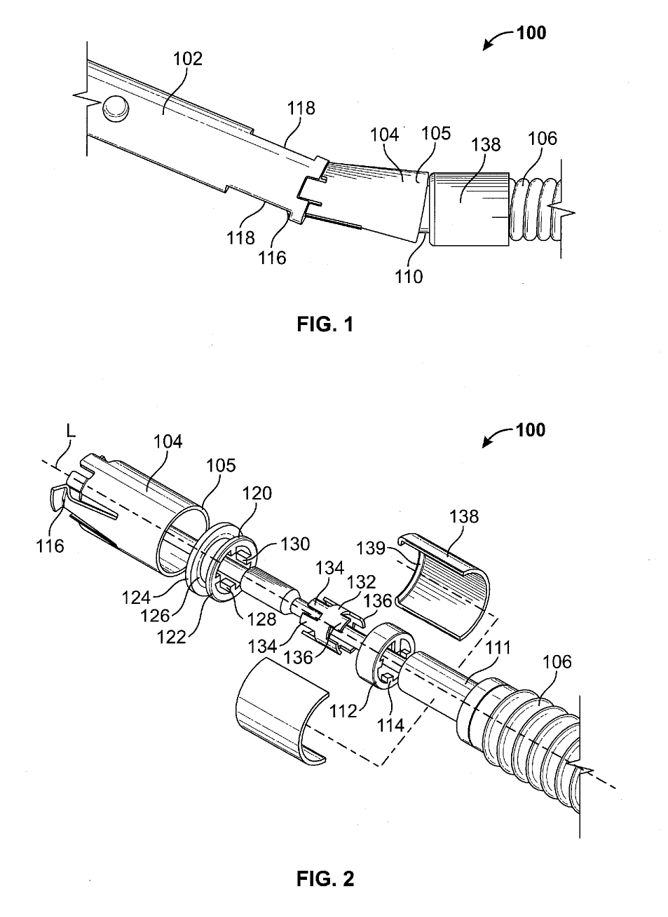

As shown in FIGS. 1-3, a device 100 according to an exemplary embodiment of the invention extends longitudinally from a proximal end including a handle accessible to a physician or other user in an operative configuration to a distal end which, in use, is inserted into a living body to a site adjacent to target tissue. Except as specifically pointed out, the device 100 according to this embodiment is formed substantially similarly to the device described in U.S. application Ser. No. 12/107,559 entitled “Single Stage Hemostasis Clipping Device” filed on Apr. 22, 2008 to Cohen et al., hereinafter referred to as “the '559 application”, the entire disclosure of which is incorporated herein by reference thereto. The end effector according to this embodiment comprises a clip containing capsule 102 attached to a bushing 104 at a proximal end thereof. The bushing 104 is connected to a flexible member 106 extending to a handle 101 configured to remain outside the body in an operative configuration. The flexible member 106 is preferably sized for insertion through a working channel of an endoscope (i.e., with an outer diameter less than an inner diameter of the working channel).

In an exemplary embodiment, the flexible member 106 is formed as an elongated coil defining a hollow channel 108 extending therethrough. A rotation tube 110 extends longitudinally through the hollow channel 108 and extends distally therepast by a predetermined distance to a clip (not shown) coupled to the distal end thereof. In an exemplary embodiment, the rotation tube 110 may be formed of one or more of Nitinol, Inconel, carbon-filled PEEK, glass-filled PEEK and any other biocompatible material having similar column strength, torsional stiffness and longitudinal flexibility. Actuation of the clip (not shown) may be performed by a control wire (also not shown) extending through the rotation tube 110, as will be described in greater detail hereinafter. An outer diameter of the rotation tube 110 may be substantially similar to an inner diameter of the flexible member 106. A portion 111 of the rotation tube 110 extending past the distal end of the flexible member 106 is dimensioned to receive a rotation tube restraint 112 thereover. The restraint 112 is formed as a substantially cylindrical hollow element having a substantially smooth outer surface and a non-smooth inner surface. Specifically, an inner wall of the restraint 112 comprises one or more protrusions 114 configured to receive a driver 132, as will be described in greater detail later on.

In an exemplary embodiment, the bushing 104 includes tabs 116 adjacent a distal portion thereof, the tabs 116 being configured to deflect radially outward when a bushing support (not shown) applies an expanding pressure thereto. Specifically, the bushing support (not shown) provided within the bushing 104. The exemplary bushing support (not shown) according to the invention may be formed as slidable element movable along a longitudinal axis of the bushing 104 to permit movement of the tabs 116 between and expanded configuration and a biased, non-expanded configuration. In an exemplary embodiment, the bushing support (not shown) is formed with a non-cylindrical shape configured to engage the tabs 116 in an operative configuration. A substantially cylindrical drive element 120, which may be considered in this embodiment a bushing base, is received at least partially within the distal portion of the bushing 104. In an operative configuration, the drive element 120 may be welded or otherwise permanently affixed to a proximal end 105 of the bushing 104. The drive element 120 may be configured and dimensioned to be partially encapsulated within a socket 138 in an operative configuration to affix the bushing 104 and capsule 102 to the flexible member 106. That is, the drive element 120 is locked in position within the socket 138 to prevent longitudinal movement of the bushing 104 relative to the flexible member 106. However, in this position, the drive element 120 is still rotatable relative to the flexible member 106, as will be described in greater detail later on. Upon engagement with the bushing support (not shown), the tabs 116 engage the slots 118. Specifically, the tabs 116 are configured and dimensioned to engage slots 118 in the capsule 102 to lock the capsule 102 to the bushing 104. The tabs 116 are constructed to be biased towards a central longitudinal axis L of the bushing 104, as would be understood by those skilled in the art. The busing support (not shown) is configured and dimensioned so that, when slid within the bushing 104 to a desired position, the bushing support engages an inner surface of each of the tabs 116 maintaining the tabs 116 in a locking position in which they are engaged with corresponding slots 118 in the capsule 102. In operation, the bushing support (not shown) applies a radially expansive pressure to the tabs 116.

The drive element 120 is formed as a hollow substantially cylindrical element having a proximal rim 122, a distal rim 124 and a grooved portion 126 extending therebetween. The grooved portion 126 is configured and dimensioned to engage a lip 139 provided on a distal end of the socket 138. As those skilled in the art will understand, a reduced diameter of the proximal rim 122 permits pivoting movement of the bushing 104 relative to the socket 138. An inner wall of the drive element 120 is provided with protrusions 128 disposed around a circumference thereof configured to define a non-cylindrical channel 130 extending therethrough. The shape and position of each of the protrusions 128 is selected to permit engagement with the driver 132 having distal drive teeth 134. In an exemplary embodiment, the driver 132 may be formed of one of laser-cut metal, stamped metal, a formed metal, a carbon-filled polymer, a glass-filled polymer and any other plastic having similar mechanical properties.

In an operative configuration, a rotation tube restraint 112 having a larger outer diameter than an inner diameter of the flexible member 106 is positioned over the portion 111 of the rotation tube 110. Proximal drive teeth 136 of the driver 132 extending through the rotation tube restraint 112 between the protrusions 114 of the restraint 112 are affixed to the portion 111 of the rotation tube 110 to lock the rotation tube restraint in a desired position at the distal end of the rotation tube 110. As the outer diameter of the rotation tube restraint 112 is greater than an inner diameter of the flexible member 106, a proximal end of the rotation tube restraint 112 defines a limit to the withdrawal of the rotation tube 110 proximally into the flexible member 106. At the same time, engagement of the proximal drive teeth 136 with the protrusions 114 prevents rotation of the restraint 112 relative to the rotation tube 110. The socket 138 is provided over the restraint 112 and driver 132 to cover this arrangement.

To load a clip onto the rotation tube 110, the distal drive teeth 134 are inserted into the drive element 120 between the protrusions 128 to couple the drive element 120 to the rotation tube 110 via the driver 132. Whereas conventional devices employ a crimped or welded connection with the bushing 104, the exemplary distal drive teeth 134 according to the invention permit greater articulation of the bushing 104. In an exemplary embodiment, the bushing 104 is permitted to pivot by approximately 10° in all directions relative to the socket 138, as shown in FIG. 1. The bushing support (not shown) is then moved distally within the bushing 104 until the tabs 116 are urged radially outward to engage slots 118 in the capsule 102 to lock the capsule 102 to the bushing 104. The drive element 120 is then fixed to the proximal end 105 of the bushing 104, as described in greater detail earlier.

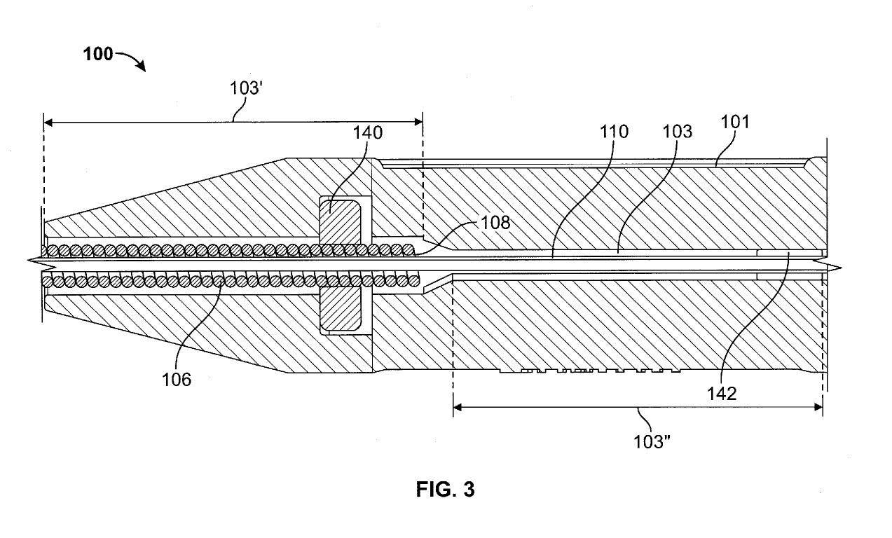

As shown in FIG. 3, the handle 101 according to the invention also permits an increased amount of rotation and movement of the flexible member 106 relative thereto. That is, whereas conventional devices require that the flexible member 106 be crimped, welded or otherwise attached to the handle 101, the apparatus according to the exemplary embodiment of the invention comprises an anchor 140 coupling the flexible member 106 to the handle 101 while permitting free rotation thereof. Thus, the flexible member 106 may be rotated as needed within the handle 101 preventing wind-up of the flexible member 106 (i.e., the buildup of torque therealong) during rotation of the handle 101, as those skilled in the art will understand. The anchor 140 is configured to lockingly engage a proximal portion of the flexible member 106. The handle 101 may be molded in two parts and the flexible member 106 with the attached anchor 140 inserted into a cavity thereof. The two parts may then be brought into alignment with one another and bonded together via any known technique (e.g., ultrasonic welding, etc.).

As also shown in FIG. 3, a proximal portion of the rotation tube 110 is attached to an anchor block 142 positioned over an outer portion thereof. In an exemplary embodiment, the anchor block 142 is welded to the rotation tube 110. It is noted however that any other suitable method of attachment may be employed without deviating from the scope of the invention (e.g., adhesive, friction fit, etc.). In another embodiment, the anchor block 142 is integrally formed with the rotation tube 110. The handle 101 comprises a longitudinal cavity 103 extending therethrough. A distal portion 103′ of the cavity 103 is configured and dimensioned to receive the flexible member 106. A diameter of a proximal portion 103″ of the cavity 103 is substantially similar to an outer diameter of the anchor block 142 to permit slidable movement of the anchor block 142 therewithin. The anchor block 142 is formed with a non-circular cross-sectional shape and dimensioned to be slidable through the proximal portion 103″ of the cavity 103. In an exemplary embodiment, the anchor block 142 has a substantially rectangular cross-sectional shape. The proximal portion 103″ is formed with a substantially circular cross-sectional shape. In operation, the anchor block 142 is longitudinally movable within the proximal portion 103″ but is prevented from rotating therewithin due to the mismatched cross-sectional shapes. This configuration permits a user to control axial movement of the rotation tube 110. Specifically, as those skilled in the art will understand, the flexible member 106 may change an effective length thereof due to bending when inserted through tissue along a tortuous path. Thus, as a length of the flexible member 106 increases, the anchor block 142 moves distally within the proximal portion 103″, so that the length of a portion of the rotation tube 110 extending distally from the handle 101 is increased. Accordingly, the exemplary anchor block 142 and proximal portion 103″ of the invention permit the flexible member 106 to be readily traversed through tissue along a tortuous path. Conversely, as the flexible member 106 is straightened, the anchor block 142 moves proximally within the proximal portion 103″ withdrawing a portion of the rotation tube 110 proximally into the handle 101.

In accordance with an exemplary method according to the invention, the flexible member 106 is inserted through an endoscope until a distal end of the device 100 comprising a clipping device or end effector (not shown) extends from the distal end of the endoscope exposed to a target portion of tissue. During insertion, the anchor block 142 automatically slides within the proximal portion 103″ to adjust a length of the rotation tube 110 extending from the handle 101. As discussed above, this length adjustment enhances the flexibility of the flexible member 106 during insertion. In another embodiment of the invention, a physician or other user may manually move the anchor block 142 within the proximal portion 103″ of the longitudinal cavity 103 as needed. The bushing 104 may also pivot relative to the socket 138 during insertion. Once inserted to the target position, a clipping device (not shown) is positioned to grasp a target portion of tissue. The bushing 104 is then separated from the capsule 102 and subsequently removed from the body, as described in greater detail in the '559 application. Specifically, once the device 100 has been inserted to the target position, the control wire (not shown) extending through the rotation tube 110 is withdrawn proximally, the proximal movement also drawing the clipping device (not shown) connected to a distal end thereof proximally. A frangible connection provided between the control wire (not shown) and clipping device (not shown) is configured to fracture or otherwise separate upon application of a predetermined load to the control wire through, for example, manipulation of an actuator. As the control wire is drawn proximally, arms of the clipping device are drawn into the capsule 102 so that contact with the capsule 102 draws distal ends of the arms toward one another, compressing tissue located therebetween. Distal portions of the arms may be configured to be wider than proximal portions thereof, defining a maximum extent to which the arms may be drawn into the capsule 102. Thus, as the control wire is drawn proximally and the distal ends of the arms approach one another, the force required to compress tissue gripped thereby applies a load to the control wire. After the arms have been drawn into the capsule 102 to the maximum extent, operating the actuator to draw the control wire further proximally applies an increasing amount of tension to the control wire and, consequently, to the frangible connection, causing the frangible connection to fail and separate the control wire from the clipping device. Furthermore, failure of the frangible connection releases tabs (not shown) of the clipping device to move laterally outward to engage the slots 118 of the capsule 102 locking the clipping device closed within the capsule 102, maintaining the clipping device constrained within the capsule 102 to keep the arms closed over tissue gripped therebetween.

It will be understood by those of skill in the art that individual features of the embodiments described above may be omitted and or combined to form alternate embodiments. Furthermore, it will be understood by those skilled in the art that various modification can be made in the structure and the methodology of the present invention, without departing from the spirit or scope of the invention. For example, although the present invention has been described with respect to a clipping device, the exemplary system and method may also be used to perform biopsy procedures or any other medical procedure wherein improved rotation of a component is required in combination with a function of opening/closing a device, extending/retracting a device into tissue, etc., as those skilled in the art will understand. Furthermore, although the present invention has been described with respect to a removable capsule 102, the exemplary rotation drive mechanism according to the invention may also be used with a biopsy tool or any other medical device non-removably attached to the rotation drive mechanism. It is therefore respectfully submitted that the exemplary rotation drive mechanism according to the invention may be employed in any other medical device requiring precise rotational control without deviating from the spirit and scope of the invention. Thus, it is intended that the present invention cover the modifications and variations of this invention provided that they come within the scope of the appended claims and their equivalents.

What is claimed is:

1. A medical device, comprising:
a flexible member extending from a handle at a proximal end to a distal end sized and shaped for insertion to a target position in a living body and having a first channel extending longitudinally therethrough;
an elongated rotation tube positioned in the first channel and being rotatable relative to the flexible member; and
a rotation drive device at a distal end of the rotation tube, a proximal end of the rotation drive device being associated with the rotation tube, a distal end of the rotation drive device having a first protrusion extending distally therefrom and a drive element having a substantially cylindrical shape with a groove on an inner wall thereof mating with the first protrusion of the rotation drive device, the drive element configured to transmit rotational movement of the rotation tube to an end effector connected to a distal end thereof to rotate the end effector relative to the flexible member.

2. The device of claim 1, wherein the end effector comprises a bushing having a proximal end connected to the distal end of the drive element, a distal end and a channel extending therethrough, the bushing comprising a plurality of arms extending distally from the distal end, the arms being maintained by a bushing support in a connection configuration in which distal ends of the arms engage a capsule to couple the capsule to the flexible member, the arms being biased toward a release configuration in which engagement surfaces at distal ends thereof are retracted radially into the channel to release the capsule from the flexible member.

3. The device of claim 2, wherein the drive element is permanently attached to a proximal end of the bushing.

4. The device of claim 1, further comprising a socket formed as a substantially cylindrical shell positioned over at least a portion of an outer surface of the drive element.

5. The device of claim 2, further comprising a bushing support slidably received within the channel of the bushing supporting the first and second arms to extend radially outward when in a first configuration.

6. The device of claim 4, wherein the drive element comprises a circumferential groove on an outer wall thereof configured to lockingly engage the socket to prevent axial movement of the drive element relative to the flexible member while allowing rotational movement of the drive element relative to the flexible member.

7. The device of claim 1, further comprising a substantially cylindrical restraint permanently engaging a second protrusion formed on a proximal end of the rotation drive device, the restraint being slidably received over a distal portion of the rotation tube.

8. The device of claim 7, wherein the restraint is locked to the rotation tube by one of a friction fit, a weld, and adhesive.

9. The device of claim 1, wherein the end effector includes a clipping device, the device further comprising a control wire inserted through the rotation tube, a proximal end of the control wire being actuatable by a user and a distal end of the control wire being connected to the clipping device.

10. The device of claim 1, further comprising an anchor block positioned over an outer surface of the rotation tube within the handle, the anchor block being slidable within a predetermined portion of the handle to affect longitudinal movement of the rotation tube relative to the handle.

11. The device of claim 10, wherein the anchor block is configured and dimensioned to prevent rotation of the rotation tube relative to the handle.

12. The device of claim 1, further comprising a coil anchor attached to an outer surface of the flexible member and locking an axial position of the flexible member relative to the handle.

13. A system for performing a medical procedure, comprising:
a flexible member extending from a handle at a proximal end to a distal end sized and shaped for insertion to a target position in a living body and having a first channel extending longitudinally therethrough;
an elongated rotation tube positioned in the first channel and being rotatable relative to the flexible member;
a rotation drive device at a distal end of the rotation tube, a proximal end of the rotation drive device being associated with the rotation tube, a distal end of the rotation drive device having a first protrusion extending distally therefrom;
a drive element having a substantially cylindrical shape with a groove on an inner wall thereof mating with the first protrusion of the rotation drive device, the drive element being configured to transmit rotational movement of the rotation tube to a medical device connected to a distal end thereof, wherein the rotation tube is rotatable relative to the flexible member; and
an end effector attached to a distal end of the drive element.

14. The system of claim 13, wherein the end effector comprises a bushing having a proximal end coupled to the flexible member, a distal end releasably coupled to a capsule and a channel extending therethrough, the bushing comprising a plurality of arms extending proximally from the distal end, the arms being maintained by a bushing support in a connection configuration in which distal ends of the arms engage a capsule of the end effector to couple the capsule to the flexible member, the arms being biased toward a release configuration in which engagement surfaces at distal ends thereof are retracted radially into the channel, the bushing being configured to removably engage the first protrusion of the rotation drive device so that the bushing is pivotable relative to a longitudinal axis of the rotation drive device.

15. The system of claim 14, wherein the drive element is permanently attached to a proximal end of the bushing.

16. The system of claim 14, further comprising a socket formed as a substantially cylindrical shell positioned over at least a portion of an outer surface of the drive element.

17. The system of claim 14, further comprising a bushing support slidably received within the channel of the bushing, the bushing support, in a first configuration, supporting the first and second arms in a radially outward position to lock the capsule to the bushing.

18. The system of claim 16, wherein the drive element comprises a circumferential groove on an outer wall thereof, the circumferential groove being configured to lockingly engage the socket to prevent axial movement of the drive element relative to the flexible member while allowing rotational movement of the drive element relative to the flexible member.

19. The system of claim 13, further comprising a substantially cylindrical restraint permanently engaging a second protrusion formed on a proximal end of the rotation drive device, the restraint being slidably received over a distal portion of the rotation tube.

20. The system of claim 19, wherein the rotation tube restraint is locked to the rotation tube by one of a friction fit, a weld, and adhesive.

21. The system of claim 13, wherein the end effector includes a clipping device, the device further comprising a control wire inserted through the rotation tube, a proximal end of the control wire being actuatable by a user and a distal end of the control wire being connected to the clipping device.

22. The system of claim 13, further comprising an anchor block positioned over an outer surface of the rotation tube within the handle, the anchor block being slidable within a predetermined portion of the handle to affect longitudinal movement of the rotation tube relative to the handle, wherein the anchor block is configured and dimensioned to prevent rotation of the rotation tube relative to the handle.

23. The system of claim 13, further comprising a coil anchor attached to an outer surface of the flexible member and locking an axial position of the flexible member relative to the handle while facilitating rotation of the flexible member relative to the handle.

24. A method for deploying a medical device in a living body, comprising the steps of
advancing a medical device to a target position in a living body, the medical device comprising a flexible member extending from a handle at a proximal end to a distal end sized and shaped for insertion to a target position in a living body and having a first channel extending longitudinally therethrough, an elongated rotation tube positioned in the first channel and being rotatable relative to the flexible member, a rotation drive device at a distal end of the rotation tube, a proximal end of the rotation drive device being associated with the rotation tube, a distal end of the rotation drive device having a first protrusion extending distally therefrom, and a drive element having a substantially cylindrical shape with a groove on an inner wall thereof mating with the first protrusion of the rotation drive device, the drive element being configured to transmit rotational movement of the rotation tube to a medical device connected to a distal end thereof, wherein the rotation tube is rotatable relative to the flexible member; and
performing a target medical procedure using the medical device connected to the drive element.

25. The method according to claim 24, wherein a tissue clipping device is connected to the drive element to perform a tissue clipping procedure.
